(19) United States
(12) Patent Application Publication
(10) Pub. No.: US 2010/0217154 A1
(43) Pub. Date: Aug. 26, 2010

(54) AUTOMATED BLOOD DRAW SYSTEM

(75) Inventors: Vivek R. Deshmukh, Vienna, VA (US); Neil R. Crawford, Tempe, AZ (US)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(73) Assignee: Catholic Healthcare West (d/b/a) St. Joseph's Hospital and Medical Center, Phoenix, AZ (US)

(21) Appl. No.: 12/393,669

(22) Filed: Feb. 26, 2009

(51) Int. Cl. A61B 5/00; A61B 19/00

(52) U.S. Cl. 600/575; 604/403

(57) ABSTRACT

An automated blood draw system operates in conjunction with an arterial or venous line. The aspiration mechanism allows the rate of aspiration, volume of aspirate, and the time interval of aspiration to be predetermined. Blood can be collected in sequential collection vials for subsequent analysis of a given laboratory parameter, or delivered directly to integrated analysis devices. While a predetermined volume of aspirate can be wasted, excessive aspiration is prevented by monitoring waste obtained in a collection receptacle. A flush system maintains the patency of the line without contamination of the specimen.

### FIELD OF THE INVENTION

The invention relates to devices used to secure blood samples from humans and animals for purposes of medical studies and patient care. More specifically the invention relates to automated blood drawing devices.

### BACKGROUND OF THE INVENTION

Periodic sampling of blood is important in a number of applications including applications related to medical studies and in monitoring patient progress and/or overall health. For example, it is often desirable to determine blood glucose levels over time after a meal in order to determine the efficacy of the body in metabolizing glucose, especially as it relates to diabetic care. Traditionally, blood drawn for the purposes of monitoring blood parameters has been done manually. In a hospital or other research or medical environment, a phlebotomist will manually draw blood by accessing a port on an existing venous or arterial line by inserting a needle in a shunt and drawing blood out using a syringe. In order to best assess the patient's health and/or to make the best study of blood and the body systems being analyzed, blood is often drawn at particular intervals known as time-points. When the blood sampling time-points are spread out, it is possible to manually draw blood, with a needle and syringe, without the need to pre-establish a blood line with an access port.

In many applications, the time-points needed for periodic blood sampling is large and blood is sampled frequently. In these cases, manual sampling of blood has numerous disadvantages. Often, manual sampling relies on a healthcare professional that has additional responsibilities besides sampling blood from the patient. In these cases the risk that a time-point sampling could be delayed or missed entirely is high. However, to avoid missing a time-point sample one or more full time attendants are required. This is an expensive and labor intensive requirement.

Even where the blood drawing technician timely arrives to sample blood, the temporal resolution of the time-point sampling is low. It is difficult for the technician to accurately determine the exact time that the blood was drawn, and in some cases the difference between the actual time-point sampling versus the desired time-point sampling may vary, for example, by tens of seconds to several minutes. With frequent sampling, such variance is counterproductive to the tests being performed.

It is therefore an object of the present invention to provide an improved system for obtaining periodic time-point sampling of blood so as to, for example, ease the labor requirements of time-point blood sampling and to significantly reduce or eliminate inherent error in manual blood sampling performed according to the current methodology.

### SUMMARY OF THE INVENTION

It is an object of the present invention to provide for an improved automated blood drawing apparatus. The improved automated blood drawing system allows for accurate and efficient sequential sampling of blood with reduced risk of contamination and ease of use.

For the purposes of obtaining periodic blood sampling from a patient or research participant, in a first embodiment of the device of the present invention, a 3-way valve assembly is incorporated into a venous or arterial line in close proximity to a patient. The valve assembly is comprised of a first, second and third port. The venous or arterial line is connected to a first port of the valve assembly and an isotonic saline source is connected via a fluid line to the second port of the valve assembly. The first and second ports are thereby configured as fluid entry points into the valve assembly. The third port is attached to aspiration tubing for the purpose of draining the valve assembly into, either a sample collecting receptacle or into a waste receptacle, as will be described below. Arterial or venous blood or saline solution may pass through the valve assembly and enter a fluid line connected to the third port of the valve assembly. The valve assembly is configured to alternatively inhibit the flow of blood or the saline solution depending on the valve assembly setting.

In one embodiment of the invention, the valve assembly is a commercially available 3-way stopcock assembly. The 3-way stopcock assembly may be manually controlled; however, automated control is preferred and provided for in embodiments of the present invention. Automated control may be accomplished, in one embodiment, by a rotary servo motor clamped to a stopcock assembly comprised of the 3-way stopcock and a durable holding device or base. The 3-way stopcock is used to control the flow of fluids from a set of tubes attached, respectively, to the source of blood and to a source of flushing solution.

As will be understood by those having ordinary skill in the art, the automated or manual control of the valve assembly as configured in one embodiment will allow for the valve be used to open and/or close, alternatively, two separate positions (blood and flushing solution) in the system. Therefore, when the valve assembly is connected to tubing as described above and the stopcock is turned to a first position, either manually or through automation, saline solution will be drawn from its source, through the stopcock from the fluid line attached at the second port and into aspiration tubing attached at the third port of the valve assembly. Alternatively, when the stopcock is in a second position, saline solution is prohibited from flowing through the valve body and into the aspiration tubing. Instead, blood will flow from the arterial or venous line, though the valve body and into the aspiration tubing. It will be understood by persons having ordinary skill in the art that a stop position can be included in the valve assembly or that a separate valve can be installed upstream of the main valve assembly in the saline solution line such that the flow of fluid can be stopped completely as needed.

Fluid flow through the plurality of fluid lines is controlled by an infusion pump. Activation of the infusion pump results in fluid flow from the venous or arterial line or from the saline source depending on the setting of the valve assembly. In a preferred embodiment of the invention, the infusion pump is pre-programmed for a specific fluid flow rate, to allow for a specific volume of fluid and/or to operate for a specific period of time. In this way, the healthcare professional can predetermine the volume of blood to be drawn from a patient at a specific blood sampling time-point.

The infusion pump used in such embodiments acts in coordination with an automated control system for the valve assembly. Coordination of the infusion pump and automated valve assembly may be accomplished via serial port programming of the infusion pump and valve assembly control. For example, PC based systems used to control anesthetic drug infusions have been adapted for use with a variety of commercially available medical infusion pumps. Alternatively, the infusion pump may be independently operated by a relay switch controlling power to the infusion pump while the valve assembly is manually or independently automatically operated.

For example, when a sampling of blood is desired, the valve assembly is automatically set to allow blood from the venous or arterial line to flow through the valve assembly and into the aspiration tubing. When the desired amount of blood has been obtained, the valve assembly may be automatically programmed to inhibit flow from the arterial or venous line and to allow fluid flow from the saline source into the aspiration tubing. Flushing of the aspiration tubing following blood sampling is desired. Once flushing of the aspiration tubing has been obtained, the infusion pump is programmed to shut off until the next scheduled blood sampling time-point.

Blood flowing into the aspiration tubing is collected for simultaneous or subsequent analysis of a given blood parameter or for blood drug concentration. Blood may be collected upon exit from the aspiration tubing in a blood collecting vial. Placement of the blood vial in the stream of the blood exiting the aspiration tubing is accomplished automatically via a commercially available fraction collector suitable for the purpose. Alternatively, blood may be collected in a bolus in heat sealable tubing. Date and time stamping of the bolus identifies the samples for subsequent analysis.

Appropriate safety features are preferably incorporated into the blood drawing apparatus. In those applications where blood exiting the aspiration tubing flows into an open vial, introduction of air into the arterial or venous line is of particular concern. To avoid the unwanted introduction of air, prior flushing of the aspiration tubing prior to a given sampling may be accomplished. Alternatively, an infusion pump may be incorporated with an internal sensor able to detect air entering the fluid lines. Other safety features, such as pressurized expulsion of blood from the aspiration tubing may be used independently or in coordination with other safety features of the system.

Malfunction and erroneous programming of the automated blood drawing apparatus is of particular concern as it may result in excessive pumping of venous or arterial blood from the a patient, or infusion of excessive saline into the venous or arterial line attached to the patient. A float sensor may be incorporated into an overflow tank so as to monitor excessive wasting of blood or saline flowing from the aspiration tubing. An alarm may be activated when the waste tank contents reach a predetermined level and power from the infusion pump may be automatically cut. Alternatively, an optical sensor may be incorporated at a desired location in at least one of the plurality of fluid lines so as to detect and calculate the volume of blood flowing through the tubing at a given sampling time. Once the volume exceeds a predetermined limit the user is notified or the system may be programmed to automatically shut off. Other sensing devices may be used independently or in addition to the safety features already described, such as mechanical, ultrasonic, or other acceptable flow sensing technologies.

An automated blood drawing apparatus consistent with the present invention may be adapted for use in systems currently established for manual blood drawing and monitoring. For example, manual systems have been developed for simultaneous monitoring of blood pressure in between blood sampling. These systems may be successfully adapted utilizing the automated features described herein.

Other modifications and improvements of currently available and described devices will become apparent to those skilled in the art from the detailed description of the invention below. The current invention is not limited by the specific and preferred embodiments described herein.

### BRIEF DESCRIPTION OF THE DRAWINGS

Further objects of the invention, together with additional features contributing thereto and advantages occurring therefrom, will be apparent from the following description of the invention when read in conjunction with the accompanying drawings; wherein:

FIG. 1 depicts a schematic representation of a single time-point sampling of blood by an automated blood drawing apparatus according to a specific embodiment of the present invention;

FIG. 2 depicts a schematic representation of the blood collection vials on a carousel-type device and a waste collector all used in association with a specific embodiment of the present invention;

FIG. 3 depicts a specific embodiment of the automated blood draw device incorporated into an arterial line pre-established to monitor blood pressure;

FIG. 4 depicts a specific embodiment of the automated blood draw device utilizing optical sensors and a timing element to improve efficiency of the device;

FIG. 5 depicts a specific embodiment of the automated blood draw device wherein coordination of apparatus components is accomplished via a single computer;

FIG. 6 depicts a specific embodiment of the automated blood draw device wherein sampled blood is collected in a bolus of pliable material;

FIG. 7 depicts another specific embodiment of the automated blood draw device wherein sampled blood is collected in a bolus of pliable material.

### DETAILED DESCRIPTION OF THE INVENTION

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiments illustrated. It should be further understood that the title of this section of the specification, namely “Detailed Description of the Invention”, relates to a requirement of the United States Patent Office, and does not imply, nor should be inferred to limit the subject matter disclosed herein.

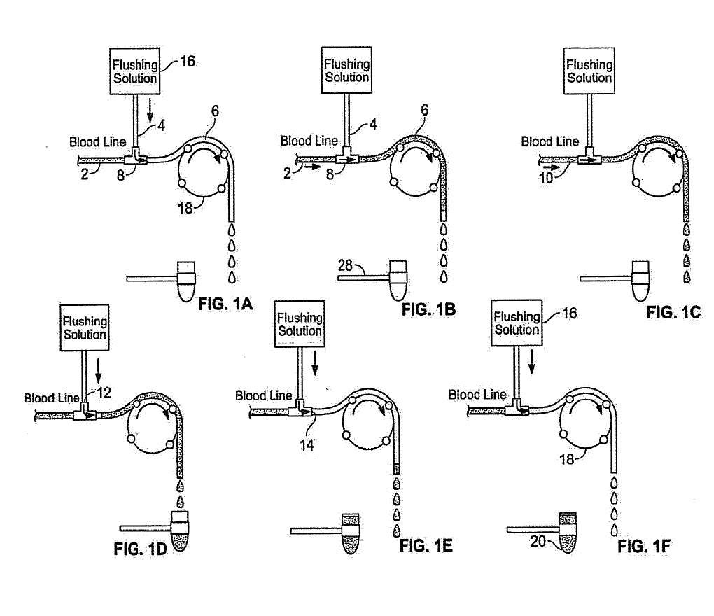

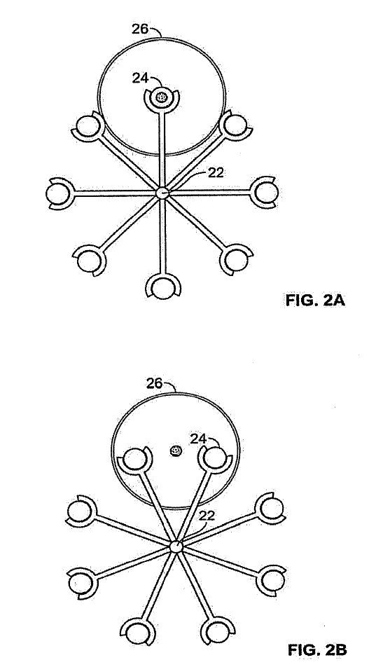

Referring to FIG. 1, In a particular embodiment of the present invention a valve assembly is comprised of a 3-way stopcock 8, a solid base and a rotary servo motor, all of which are known to persons having ordinary skill in the art. Disposable 3-way stopcocks appropriate for the patient environment are commercially available and preferred for their ease of use. The 3-way stopcock is provided with means for selectively determining the position of an internal valve within the stopcock body to allow fluid flow through the stopcock body from one of two input ports and out of a third port.

A solid base, such as of metal or hard plastic, is provided to receive and securely clamp the stopcock body. Ideally, placement of the stopcock valve assembly at the base is accomplished without tools. For example, the stopcock assembly may be placed by press fitting the assembly to the base. The solid base may also be associated with means providing easy access by a health care professional to the 3-way stopcock. Additionally, a rotary servo motor may be clamped to the stopcock body and base to allow automated operation of the internal valve so as to determine at least two positions of the valve. The rotary servo motor in conjunction with the 3-way stopcock and solid base comprises the valve assembly.

It will be apparent to one skilled in the art that the invention is not limited to the specific valve assembly described. For example, the 3-way stopcock may be replaced in appropriate applications with a 1-way or 4-way stopcock incorporated into the previously described valve assembly. Alternatively, T-branches as commonly known in the art may be used to interconnect tubing. A T-branch is comprised of a first, second and third port that can accept the blood line 2, flushing line 4 and aspiration line 6 of FIG. 1 respectively. In lieu of the valve apparatus of the stopcock, multiple blunt pinchers may be used to facilitate or inhibit fluid flow in the plurality of fluid lines. Before use, the interconnected tubing would be pressed into the jaws of the pinchers. In one embodiment of the invention, servo motors may be used to control the pinchers, enabling one or more sections of tubing to be pinched closed while simultaneously releasing one or more sections of tubing, thereby facilitating fluid flow. Other modifications of the valve assembly consistent with the spirit and scope of the present invention will be obvious to those skilled in the art. The preceding is included for completeness of the description and while numerous elements described are not shown in the illustration, persons having ordinary skill in the art will understand the use and placement of such elements.

Referring now to FIG. 1, in one particular embodiment of the invention utilizing a valve assembly with a 3-way stopcock, the 3-way stopcock 8 valve assembly is associated with a patient blood line 2. The blood line 2 is connected at an origin position to a patient, in a manner well known to medical and research professionals, and at a terminal position to a first port 10 of the 3-way stopcock 8. Preferably, the length of the blood line 2 is kept small so the total volume of blood required to fill the blood line is minimized and excessive blood waste from the patient is avoided. In an alternative embodiment of the invention, the blood line is a previously established venous or arterial line wherein the valve assembly is incorporated into the venous or arterial line at a position in close proximity to the patient.

A fluid line 4 is connected at an origin position to a flushing solution source 16 and at a terminal position to a second port 12 of the 3-way stopcock 8 valve assembly. It will be understood by persons having ordinary skill in the art that the flushing solution will be utilized to cleanse the valve and aspiration tubing preceding each blood sampling as will be described in detail below. In a specific embodiment of the invention, the flushing solution source 16 attached to the origin of the fluid line 4 is comprised of an isotonic saline solution. In some applications it may be desirable to utilize an isotonic flushing solution with additives, such as heparin, to better effectuate clearing of the automated blood apparatus of blood in between sampling. The flushing solution is utilized in applications according to the invention so as to flush the stopcock valve and the aspiration tubing after a given sampling of blood and to further ensure fluid flow through the stopcock valve and the plurality of fluid lines does not become obstructed.

Finally, aspiration tubing 6 is connected at an origin position to a third and final port 14 of the 3-way stopcock 8 valve assembly. The terminus of the aspiration tubing allows for elimination of fluid originating from either the blood line 2 or the fluid line 4 into appropriate collecting means or into a waste collection tub 26 (see FIG. 2). Where it is desired to incorporate the valve assembly into a pre-existing venous or arterial line, the pre-existing line is cut and the cut termini of the venous or arterial line are attached at the first and third ports of a 3-way stopcock as previously described, forming the blood line and the aspiration tubing respectively. The flushing line 4 is then established as previously described.

Referring now to FIG. 1A, when the 3-way stopcock is manually or automatically set to a first position the flushing solution 16 is drawn into the flushing line 4, through the stopcock 8 and into the aspiration tubing 6 attached at the third port of the stopcock. Flushing solution is prohibited from entering the blood line 2 attached to the first port of the stopcock. Alternatively, as shown in FIG. 1B, when the 3-way stopcock is set to a second position blood is drawn into the blood line 2, through the stopcock 8 and into the aspiration tubing 6. Blood is prohibited from flowing into the fluid line 4 attached to the second port of the stopcock when the stopcock is in either the first or second position. Flushing solution and blood passing through the stopcock body and into the aspiration tubing is collected, wasted and/or analyzed as described in detail herein.

Referring generally to FIG. 1, fluid flow from the flushing solution source 16 or from the blood line 2 is controlled by an infusion pump 18. When the infusion pump is inactive, fluid flow through the stopcock body 8 is inhibited. Upon activation of the infusion pump, fluid flows through the stopcock body 8 and into the aspiration tubing 6. Activation of the infusion pump may be manually effectuated. Alternatively, in a preferred embodiment of the invention, activation of the infusion pump 18, the rate of fluid flow into the aspiration tubing 6, the volume of aspirate, and/or the time interval of aspiration are pre-programmed and automated. In one embodiment of the invention, an analog infusion pump operable by a relay switch controls power to the infusion pump. Alternatively, serial port programming of the infusion pump 18 can be used to control fluid flow through the stopcock body 8 and into the aspiration tubing 6. For example, PC based systems used to control anesthetic drug infusions have been adapted for use with a variety of commercially available medical infusion pumps and may be successfully adapted for use with the present invention.

According to one embodiment of the invention, blood is collected upon exit from the aspiration tubing 6 in a vial 20 of an appropriate size for the application. Preferably, vial placement in the blood stream is accomplished automatically. For example, FIG. 1 depicts a linear actuator 28 that may be used to place a vial 20 in one of two positions. A first position, shown in FIG. 1A, places the vial 20 out of the stream of fluid flowing from the aspiration tubing 6. When the linear actuator 28 is in this position, fluid flowing from the aspiration tubing 6 is collected in a waste receptacle. A second position of the linear actuator 28, shown for example in FIG. 1D, places a collection vial 20 in the path of blood flow and allows for the collection of blood exiting from the aspiration tubing 6.

Alternatively, it may be desirable to sequentially obtain blood from the aspiration tubing 6 in multiple collection vials. An apparatus, such as a fraction collector able to hold multiple vials and sequentially place them in a stream of blood flowing from the aspiration tubing may be used. In one embodiment of the invention demonstrated at FIG. 2, a rotating tray 22 capable of holding a plurality of vials 24 is used for the purposes of obtaining sequential blood samples automatically and without manual intervention. Flushing solution and stagnant blood exiting the aspiration tubing 6 is collected in a waste tub 26 located beneath the rotating tray 22. When the waste fluid has been fully cleared from the aspiration line 6, the rotating tray automatically places the next available collection vial 24 into the blood stream thereby collecting the desired time-point blood sample. In one specific embodiment of the invention, the rotating tray may be coupled with a point-of-care analyzer such as an ACT monitor to analyze blood parameters in the collected sample. While the sample is being analyzed, the adjacent vial is positioned to gather the next sample. This system allows for automation of several samples sequentially. The ACT analysis cartridge may be changed by the health care provider at change of shift or at set intervals.

Referring again to FIG. 1, a time-point sampling of blood from a patient according to one embodiment of the invention is shown. The 3-way stopcock 8 valve is manually or automatically set to a first position to allow flushing solution 16 to flow into the aspiration tubing 6. The infusion pump 18 is activated manually or automatically to completely flush the stopcock body 8 and the aspiration line 6, as shown at FIG. 1A. Flushing solution exits from the aspiration tubing 6 into a waste receptacle. Adequate flushing of the aspiration tubing allows for accurate blood sampling and prevents contamination of the aspiration line.

Referring now to FIGS. 1B and 1C, once the aspiration line 6 has been adequately flushed, the stopcock valve is manually or automatically set to a second position, thereby allowing blood to flow through the stopcock body 8 and into the aspiration tubing 6. The infusion pump 18 is activated manually or automatically to allow blood from the blood line 2 to enter the aspiration tubing 6. The blood line 2 will be filled with stagnant blood left over from the previous time-point blood sampling and must be eliminated from the system before the time-point blood sample is collected, as shown in FIG. 1C. Likewise, flushing solution filling the stopcock body and the aspiration tubing must be eliminated from the system, as shown in FIG. 1B. Stagnant blood and flushing solution are eliminated from the system and collected in a waste container. The linear actuator 28 may be manually operated or automated in conjunction with the infusion pump 18 to ensure the vial 20 remains in a first position out of the stream of fluid exiting the aspiration tubing 6 until such time that the flushing solution and stagnant blood have cleared the system.

In one embodiment of the invention, a second infusion pump may be placed along the blood line 2 between the stopcock 8 and the patient to allow for flushing of the blood line 2 in between blood sampling. The infusion pump is activated at the end of a time-point blood sampling either before or after the aspiration tubing 6 has been flushed. Appropriate flushing solution 16 flows through the valve body and into the blood line 2, and toward the patient. The infusion pump may be manually or automatically operated to ensure excessive flushing solution does not enter the blood line 2 and thereby the patient. An appropriate valve assembly is selected in systems calling for flushing of the blood line between time-point blood sampling and other modifications apparent to one skilled in the art are within the scope of the invention.

Referring now to FIG. 1D, the linear actuator 28 is manually or automatically activated to move the collection vial 20 into the stream of blood exiting the aspiration tubing 6. A time-point sampling of blood is collected manually or automatically. In a preferred embodiment of the invention, the blood sample is collected automatically. The system is pre-programmed to calculate the amount of blood flowing into the aspiration tubing from the patient. System dependent parameters that may be entered by a technician include the length of the blood line 2, the length of the aspiration tubing 6, the infusion pump 18 speed or the volume rate of fluid flowing through the aspiration tubing 6 and/or the volume of blood to be sampled at each time-point. In certain applications, it may be desirable to deliver a quantity of flushing solution to the collection vial, for example, to deliver an additive such as heparin present in the flushing solution. In this manner, vials pre-packaged containing heparin or any other desired additive may be obviated.

Once the desired volume of blood for a time-point sample has entered the aspiration tubing 6, the stopcock 8 valve is set to a first position to allow flushing solution 16 to enter the stopcock body and flow into the aspiration tubing 6. In this manner, the total volume of blood drawn from the patient at each time-point sampling is carefully calculated and the system may be programmed to minimize wasting. Minimization of wasting is particularly important where a number of time-point blood samples are required over a relatively short period of time.

After the desired volume of blood has been collected in the collection vial 20, the linear actuator 28 moves the collection vial out of the stream of blood exiting the aspiration tubing 6, as shown in FIGS. 1E and 1F. To provide for accurate blood sampling and to prevent contamination the aspiration tubing must be flushed between sequential blood draws. The stopcock 8 and aspiration tubing 6 are completely flushed with flushing solution 16. In one embodiment of the invention, the automated blood draw system is programmed to allow the residual blood and a predetermined amount of flushing solution to pass through the aspiration tubing 6 and into the waste collection container. Flushing is coordinated to avoid collection of flushing solution in the blood collection vials and to minimize blood waste.

Once the aspiration tubing 6 has been completely flushed, the infusion pump 18 is manually or automatically shut off to inhibit the flow of fluid through the system. The automated blood draw system is inactive until the next scheduled time-point blood sampling is desired. Blood collected in the collection vial 20 is manually or automatically stored or processed and a new collection vial prepared for the next sampling.

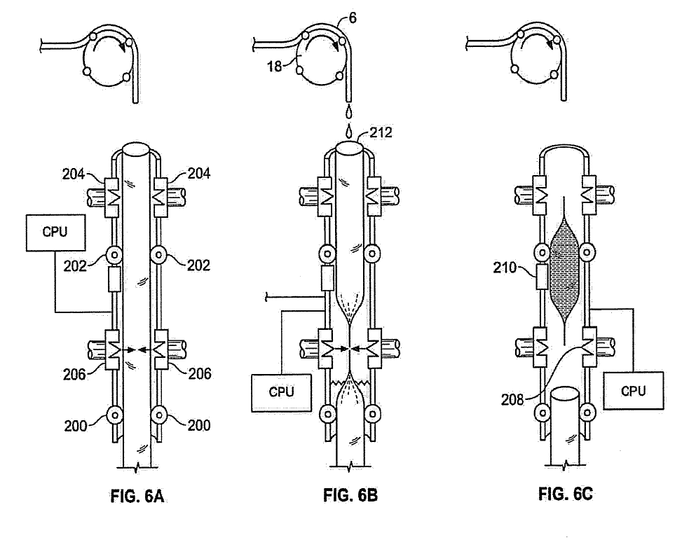

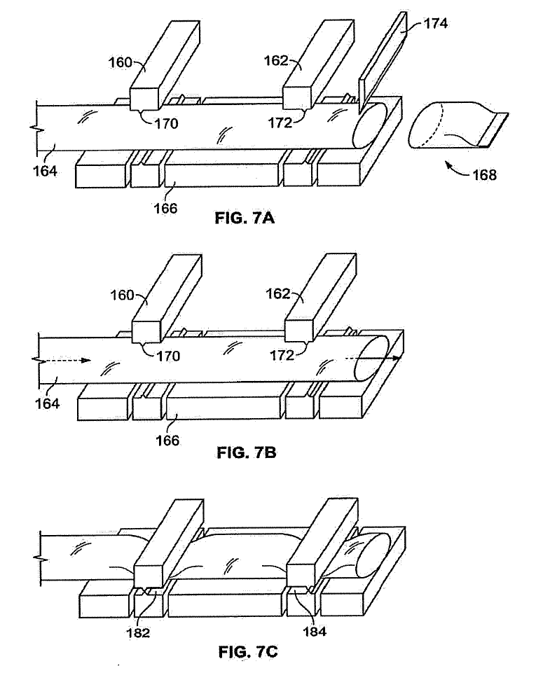

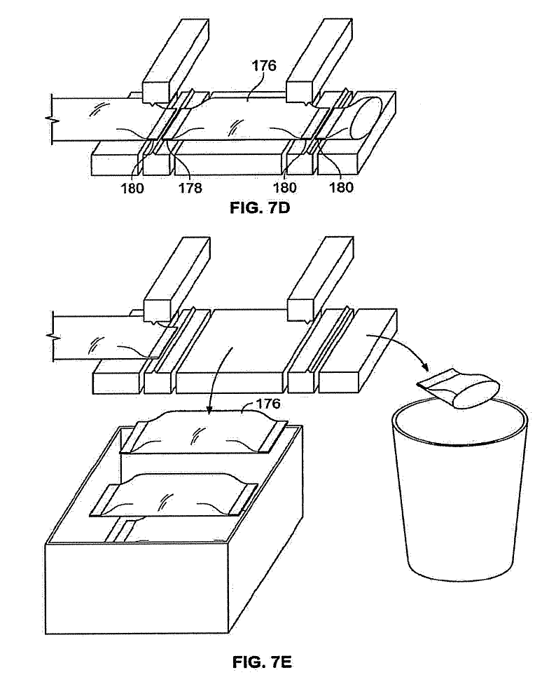

A specific embodiment of the invention has been described whereby time-point blood samples are collected in collection vials 20. Alternatively, a time-point blood sample may be collected as a bolus within a heat-sealable sheath of pliable tubing as shown in FIGS. 6 and 7. Referring to FIG. 7, blood exiting the aspiration tubing is introduced into the pliable collection tubing material 164. Once the desired volume of blood has entered the collection tubing, heating and pressure means, for example heated wires and pressure rollers, are provided for heat-sealing at a first 182 and second 184 position along the tubing length, thereby creating a bolus 176 of blood of the desired volume. A time-point sample identifying stamp may be pressed into a crimped portion 178 of the pliable material, Air may be evacuated from the bolus prior to heat sealing to ensure the integrity of the sample prior to processing. The heat sealed bolus is then cut from the remaining tubing utilizing, for example, a plurality of cutting elements 170 and 172 adjacent the heating elements 160, 162. Flushing of the aspiration tubing may then proceed as previously described and the tubing material advanced for a subsequent sampling.

In one embodiment of the invention, referring to FIG. 6, an automated device for pinching, cutting, and advancement of the pliable collection tubing containing a bolus of blood may utilize first 202 and second 200 rolling elements positioned adjacent heated wires 204, 206. The heated wires may be pre-spaced to an appropriate separation to achieve the desired bolus volume. The heated wires 204, 206 are capable of pressing in on the pliable tubing 212 while heating the material so as to seal the tubing material upon cooling. A guillotine 208 for cutting the heat sealed bolus is provided adjacent the second roller means. Preferably, when the collected bolus has been sealed from the unused collection tubing, means are provided 210 for time marking either directly to the tubing or to a label attached to the tubing the time at which the blood sample was sealed and any other identifying information that may be helpful when later handling the bolus. Collection tubing used in accordance with the invention should be supplied with sufficient excess material to allow for collection of the desired number of blood samples without the risk of running out of the pliable collection tubing.

In some applications, it may be desirable to further automate the device to allow for immediate analysis of one or more blood parameters. For example, it is often necessary to perform real-time evaluation of the Activated Clotting Time (ACT) of a given blood sample. In current practice, a health care provider is often required to manually recover a collected sample of blood so as to perform real-time ACT analysis. The present invention may be successfully practiced to automate the ACT analysis so as to provide faster and more efficient blood parameter readings.

An automated blood sampling device according to a specific embodiment of the present invention may be pre-programmed to periodically determine the Activated Clotting Time of an aspirated volume of blood. Automated means known in the art are adapted to perform ACT analysis of a blood volume collected in a collection vial as previously described and to provide real-time display of ACT. Alternatively, blood may be delivered automatically to a testing apparatus that is moved into the stream of blood exiting the aspiration tubing after a blood sample has been obtained in a collection vial. The testing apparatus is adapted to perform ACT analysis on the sample in the usual way. It will be obvious to one skilled in the art that further automation of the invention to allow for blood parameter analysis is not limited to the specific embodiments described.

The invention may also be successfully adapted for practice with an arterial line that has been established to monitor blood pressure and to allow the delivery of pressurized saline. In these applications it is possible to allow for the periodic sampling of blood while maintaining the functionality of the blood pressure monitoring system.

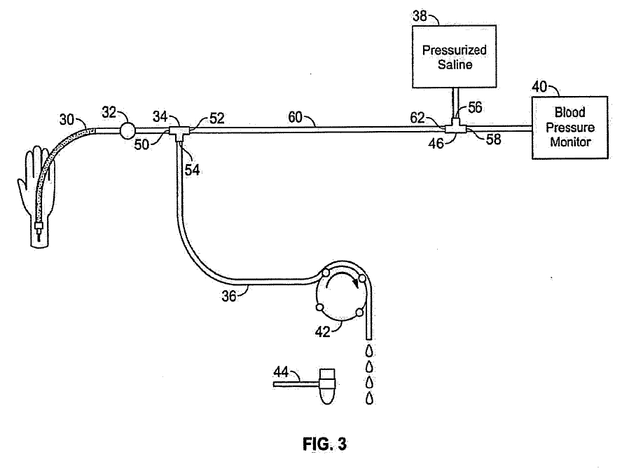

Referring to FIG. 3, a normal arterial line 30 is provided with access means in the form of a port 32 adjacent the insertion of the arterial line 30 into a patient. The port 32 provides access to the patient's bloodstream for delivery of medication. Upstream of the port 32, a 3-way stopcock 34 is inserted by cutting the pre-existing arterial line and attaching the cut termini to a first 50 and third 52 port of the stopcock body. Aspiration tubing 36 is attached to a second 54 port of the stopcock body. The stopcock 34 is inserted such that the pressurized saline source 38 and blood pressure monitoring means 40 are located upstream.

Fluid lines incorporating the pressurized saline source 38 and blood pressure monitoring means 40 into the automated blood drawing device are set up in the usual manner. For example, a second 3-way stopcock 46 receives fluid lines from the saline source 38 and blood pressure monitoring means 40 at a second 56 and third 58 port of the stopcock 46 body respectively. The transmission line 60 is attached at an origin to the third port 52 of the stopcock 34 and at a terminus to the third port 62 of the stopcock 46 receiving fluid lines from the saline source 38 and blood pressure monitoring device 40. The first 34 and second 46 stopcocks may be manually or automatically controlled, for example, utilizing rotary servo motors.

The saline source 38 connected via the upstream stopcock 46 may be used to flush the aspiration tubing 36 and optionally the arterial line 30 in between time-point blood sampling. The stopcock valves, infusion pump, and linear actuator 44 used to collect sample blood may be manually or automatically controlled. System flushing, blood collection, and wasting of flushing fluid and stagnant blood is accomplished in the manner previously described. The pressurized saline source 38 acts similarly to the flushing solution 16 of FIG. 1 when the stopcock 46 valve is set in a first position allowing pressurized saline to flow through the stopcock body and into the transmission line 60.

It will be readily apparent to one skilled in the art that pre-programming of the automated blood draw system is preferred. In one embodiment of the invention, multiple programming interfaces may be used to independently control an infusion pump, a stopcock valve assembly comprised of a servo motor and a blood fraction collecting device. User interfaces are commonly associated with commercially available servo motors, infusion pumps and fraction collectors.

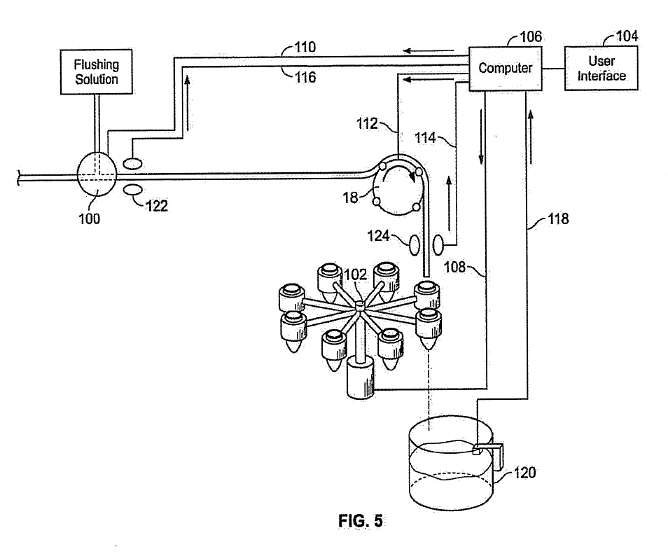

Alternatively, referring to FIG. 5, in a preferred embodiment of the invention, a single user interface 104 is provided for programming a computer 106. For example, a single computer interface 104 may be used to accept programming input to control a servo motor 100, an infusion pump 18 and vial carousel 102 according to the present invention. Appropriate system parameters are entered into the computer and a microprocessor coordinates the operation of the component parts to achieve the desired result by generating output signals 108, 110, 112. While systems with a single user interface are preferred, the present invention is not limited to single user interface systems or to systems designed for automated operation.

The computer 106 may also be adapted to receive output signals 114, 116, 118 generated by monitoring devices. Monitoring devices may include, for example, a fluid waste container 120 or fluid sensors 122, 124. The present invention is not limited to the specific monitoring devices described herein, and one skilled in the art will recognize obvious modifications that are within the scope of the present invention.

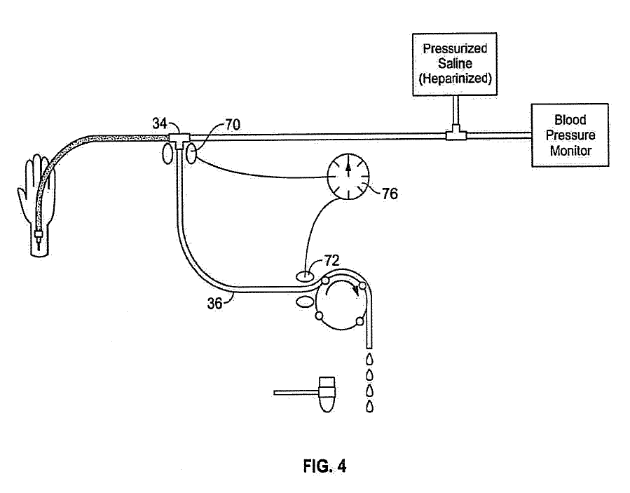

The automated blood drawing system according to the present invention may be further automated to provide for more precise measuring of blood flow through the stopcock body and into the aspiration tubing. In one embodiment of the present inventions, referring to FIG. 4, optical sensor switches are provided in cooperation with timing means and together are adapted to measure the quantity of blood passing through the aspiration tubing at a given sampling. A first optical sensor 70 is placed along the aspiration tubing 36 adjacent the valve body 34. A second optical sensor 72 is placed along the aspiration tubing 36 at a position downstream of the valve body 34 and before the open end of the aspiration tubing 36. The optical sensors are able to detect whether blood or flushing solution is flowing through the aspiration tubing 36 adjacent the respective sensor based on the absorption properties of the liquid.

The first 70 and second 72 optical sensors are provided with means for communicating with a timer 76. The timer 76 may be, for example, a mechanical timer, a digital recorder, or a computer. In a preferred embodiment of the invention, when blood enters the aspiration tubing 36 from the valve body 34 the first optical sensor 70 sends a signal to a timing computer 76, resulting in the initiation of the timing clock. When the blood reaches the second sensor 72, a signal is sent to the timing computer 76. The computer then calculates the rate of blood flow through the aspiration tubing 36 based on pre-programmed system parameters and the timing between activation of the first and second optical sensors. This information may be used by the computer to coordinate other system components resulting in efficient blood sampling. Likewise, the optical sensors are able to calculate the rate of flushing solution passing through the aspiration tubing so as to ensure adequate flushing of the line.

The information obtained from the optical sensors and delivered to the computer may also be used to generate a time stamp for a given time-point blood sampling. The exact timing of the blood draw, the volume of blood obtained, and other pertinent system parameters may be recorded to a database for future reference. Other modifications of the system utilizing optical sensors to coordinate functionality of various components within the scope of the present invention will be apparent to those skilled in the art.

Consistent with the scope of the invention, appropriate safety features may be incorporated into particular embodiments of the invention. For example, in those applications where collection blood is delivered directly into an open vial, accidental introduction of air into the arterial or venous line is a particular safety concern. Referring to FIG. 1A, to prevent unwanted introduction of air into the plurality of fluid lines, isotonic saline solution may be run through the aspiration tubing 6 before the stopcock 8 valve is set to a second position, thereby allowing blood to enter the aspiration tubing 6. In addition, the infusion pump 18 may be adapted with an internal alarm programmed to sound when air enters the aspiration tubing 6.

In another embodiment of the invention adapted to prevent air from entering the system, blood and saline may be pressure forced through the stopcock body and aspiration tubing rather than allowing sample or waste fluid to drip freely from the terminus of the aspiration tubing and into the desired collection receptacle or waste collector. A valve that opens only after exceeding a minimum pressure may be used since the infusion pump creates pressure downstream of the valve.

An additional safety consideration is a potential malfunction or erroneous programming of the automated blood draw system that may result in excessive pumping of arterial or venous blood through the aspiration tubing and into the collection container. A fluid float, such as those commonly used to indicate gas level in a closed tank may be used to monitor the level of waste collected in a waste container. An alarm may be programmed to activate when excessive fluid is collected. In an alternative embodiment of the invention, power to the infusion pumps may be cut when the fluid level in the waste container has passed a pre-determined level indicating excessive fluid waste by the system.

In another embodiment of the invention, an optical system sensitive to the difference in light absorption between clear flushing solution and opaque blood may be used to monitor when blood is being aspirated. Such a device may be placed at a point along the aspiration tubing before or after the infusion pump. The volume of aspirated blood may be calculated, based for example on the length of time the infusion pump has been operational, volume of through-flow per second for the tubing and infusion pump used, and/or on whether blood or saline was being pumped through the system during the infusion pumps operation. If this blood volume exceeds a pre-set limit of aspiration, the user would be notified and/or power to the infusion pump would be cut.

In yet another embodiment of the invention, a flow sensor may be placed around or in series with the section of tubing coming from the patient's blood line, before the intersection of the blood line with the saline line, to monitor the amount of blood flowing out of the patient. This flow sensor could be mechanical (e.g., paddle wheel), ultrasonic (e.g., Doppler), or be comprised of other accepted flow sensing technology. When total volume of blood outflow exceeds a pre-set limit of aspiration, the user would be notified and/or power to the infusion pump would be cut. Additional safety features within the scope of the present invention will be apparent to one skilled in the art.

A specific embodiment of an automated blood drawing apparatus according to the present invention has been described for the purpose of illustrating the manner in which the invention is made and used. It should be understood that the implementation of other variations and modifications of the invention and its various aspects will be apparent to one skilled in the art, and that the invention is not limited by the specific embodiments described. Therefore, it is contemplated to cover the present invention and any and all modifications, variations, or equivalents that fall within the true spirit and scope of the basic underlying principles disclosed and claimed herein.

1. An automated blood drawing apparatus for drawing blood samples at scheduled time intervals from a human or animal, comprising:
a branching element capable of transmitting fluid and engaging at least three fluid lines;
the first fluid line being capable of transmitting blood from a human or animal to the branching element;
the second fluid line being capable of delivering a flushing solution from a flushing solution source to the branching element;
the third fluid line being capable of transmitting fluid arriving at the branching element from either the first or second fluid line to collection means located at the terminus of the third fluid line;
the collection means comprising at least one collection receptacle and a waste container;
the collection means being capable of moving the collection receptacle to a first position to allow fluid exiting the third fluid line to empty into the collection receptacle or to a second position to allow the fluid exiting the third fluid line to empty into the waste container, wherein the collection receptacle is moved to a first or second position in response to an input signal;
at least one pumping means capable of initiating fluid flow through the branching element and the plurality of fluid lines upon activation, the pumping means being selectively activated or deactivated in response to an input signal;
at least one selection means for selectively inhibiting fluid flow in at least one of the fluid lines while the pumping means is activated, the selection means being capable of selective activation in response to an input signal;
wherein activation of one or more selection means allows blood to flow from the first fluid line through the branching element and into the third fluid line or allows flushing solution to flow from the second fluid line through the branching element and into either the first or third fluid line;
computer means capable of providing an input signal to the pumping means, the selection means, and the collecting means in response to system generated information;
the system generated information being produced by the computer means in response to programming information entering the computer means from at least one user interface and from at least one monitoring device;
the system generated information resulting in output signals allowing for coordination of the pumping means, selection means, and the collecting means such that efficient blood sampling occurs at scheduled time intervals without excessive blood waste.

2. An automated blood drawing apparatus according to claim 1, wherein the collection means is comprised of a fraction collector and a waste receptacle;
the fraction collector being comprised of a carousel tray and a plurality of collection receptacles;
wherein in response to an input signal, the carousel tray is moved to a first position whereby one of a plurality of collection receptacles is placed into a stream of fluid exiting the third fluid line;
wherein in response to a second input signal, the carousel tray is moved to a second position whereby the first collection receptacle is moved out of the stream of fluid exiting the third fluid line, fluid exiting the fluid line thereby being collected in a waste container located beneath the carousel tray;
wherein subsequent input signals result in the carousel tray being moved so as to place additional collection vials sequentially in and out of the stream of fluid exiting the third fluid line.

3. An automated blood drawing apparatus according to claim 1, wherein a monitoring device determines the volume of fluid in the waste receptacle;
said monitoring device being capable of generating an output signal to the computer means when the fluid level in the waste container exceeds a pre-determined level;
said output signal resulting in sounding of an alarm and deactivation of the pumping means, thereby preventing excessive blood or flushing solution aspiration.

4. An automated blood drawing apparatus according to claim 1, wherein the blood is captured in a bolus of pliable tubing.

5. An automated blood drawing apparatus according to claim 4, wherein the pliable tubing is sealed at one end and the collection means is further comprised of sealing means, cutting means and advancement means;
the pliable tubing material being capable of softening in response to an application of a specific temperature stimulus, whereby removal of the temperature stimulus results in the pliable material hardening relative to its softened state;
the sealing means being comprised of at least two heating elements, each capable of applying heat or pressure at two positions tangent to the pliable tubing, said tangents being parallel one to the other;
the advancement means being capable of advancing a section of pliable tubing into a position relative to the sealing means such that the first heating element is located at a position adjacent a cut end of the pliable tubing and the second heating element is located at position below the first heating element;
the second heating element is capable of applying heat and pressure to the pliable tubing in response to an input signal, whereby application of heat and pressure to the pliable tubing results in melting of the pliable tubing and a sealing of the tubing upon cooling;
the collection receptacle formed by sealing at the second heating element is capable of accepting fluid from the open end of the third fluid line when placed in the stream of fluid;
the first heating element is capable of applying heat and pressure to the pliable tubing of the collection receptacle in response to an input signal generated once fluid has been introduced into the receptacle, whereby application of heat and pressure to the open end of the collection receptacle results in melting of the pliable tubing and sealing of the collection receptacle, thereby creating a bolus of fluid;
the cutting means is capable of cutting the sealed bolus from the remainder of the pliable tubing;
after cutting, the advancement means is capable moving the bolus out of proximity of the sealing means and advancing additional pliable tubing into a position relative to the sealing means.

6. An automated blood drawing apparatus according to claim 4, wherein fluid exiting the third fluid tube enters a length of pliable tubing and exits into a waste receptacle;
the pliable tubing being positioned along a surface;
first and second heat sealing elements are positioned along a length of pliable tubing,
the heat sealing elements being capable of pressing the pliable tubing down on the flat surface, thereby heating the pliable material and adhering it to itself upon cooling;
the pliable tubing is sealed so as to obtain a bolus of blood in the tubing between the first and second sealed portion;
cutting means cutting the pliable tubing adjacent the sealed portion so as to free the bolus of blood from the remaining tubing material;
a cutting means further cutting the pliable tubing so as to allow fluid to flow through the remaining pliable tubing and into a waste container.

7. An automated blood drawing apparatus according to either of claims 5 and 6, wherein system generated information is affixed or stamped to the bolus.

8. An automated blood drawing apparatus according to claim 1, wherein a monitoring device is comprised of at least two optical sensors in communication with a timing means;
the first optical sensor being located at a position along the third fluid line and adjacent the branching means;
the second optical sensor being located at a position along the third fluid line downstream of the first optical sensor;
the timing means being capable of monitoring time;
the first and second optical sensors being capable of generating an output signal to the timing means in response to changes in the opacity of fluid flowing through the third fluid line;
the first output signal being generated when a change in opacity indicates either blood or flushing solution has entered the third fluid line, said output signal resulting in the timing means polling time;
the second output signal being generated when a change in opacity indicates either blood or flushing solution has reached a position along the third fluid line corresponding to the location of the second optical sensor, said output signal resulting in the cessation of time polling by the timing means, whereby the polled time may be used by the computer to generate system generated information.

9. An automated blood drawing apparatus for drawing blood samples at scheduled time intervals from a human or animal, comprising:
at least two branching elements each capable of transmitting fluid and engaging at least three fluid lines;
the first fluid line of the first branching element is comprised of a blood line being capable of transmitting blood from a human or animal to the first branching element;
the second fluid line of the first branching element being capable of delivering a flushing solution from a flushing solution source to the first branching element;
the third fluid line of the first branching element being comprised of an aspiration line being capable of transmitting fluid arriving at the first branching element from either the blood or second fluid line to collection means located at the terminus of the aspiration line;
the second branching element being incorporated upstream of the second fluid line, such that the second branching element also engages the second fluid line;
the first fluid line of the second branching element is comprised of a flushing line capable of delivering a flushing solution from a flushing solution source through the second branching element to the second fluid line;
the third fluid line of the second branching element is comprised of a monitoring line attached to a blood pressure monitoring device capable of monitoring the blood pressure of a human or animal;
the collection means comprising at least one collection receptacle and a waste container;
the collection means being capable of moving the collection receptacle to a first position to allow fluid exiting the third fluid line to empty into the collection receptacle or to a second position to allow the fluid exiting the third fluid line to empty into the waste container, wherein the collection receptacle is moved to a first or second position in response to an input signal;
at least one pumping means capable of initiating fluid flow through the branching elements and the plurality of fluid lines upon activation, the pumping means being selectively activated or deactivated in response to an input signal;
at least two selection means for selectively inhibiting fluid flow in at least two of the fluid lines while the pumping means is activated, the selection means being capable of selective activation in response to an input signal;
wherein activation of one or more selection means allows blood to flow from the blood fluid line through the first branching element and into the aspiration line or allows flushing solution to flow from the flushing line, through the second branching element, into the second fluid line, through the first branching element and into either the blood or third aspiration line, or allows the blood pressure monitoring device to monitor the blood pressure of a human or animal, the blood line, second fluid line, and monitor line forming a continuous fluid line when the blood pressure monitoring device is active;
computer means capable of providing an input signal to the pumping means, the selection means, and the collecting means in response to system generated information;
the system generated information being produced by the computer means in response to programming information entering the computer means from at least one user interface and from at least one monitoring device;
the system generated information resulting in output signals allowing for coordination of the pumping means, selection means, and the collecting means such that efficient blood sampling occurs at scheduled time intervals without excessive blood waste.
